(19) United States
(12) Patent Application Publication
(10) Pub. No.: US 2002/0151937 A1
(43) Pub. Date: Oct. 17, 2002

(54) Iontoforesis device

(76) Inventor: Yoshiyasu Ito, Tokyo (JP)

Correspondence Address: BELASCO, JACOBS & TOWNSLEY LLP, 100 CORPORATE POINTE, SUITE 330, CULVER CITY, CA, 90230, US

(21) Appl. No.: 10/091,205

(22) Filed: Mar. 5, 2002

(30) Foreign Application Priority Data
Mar. 6, 2001 (JP) 2001-061888

(51) Int. Cl. A61N 1/18

(52) U.S. Cl. 607/20

(57) ABSTRACT

A portable iontoforesis device that can be used by everybody anytime anywhere. A freely-detachable medicine container, which supplies medicine to the first electrode, is mounted on the main body of the iontoforesis device. The liquid medicine flows from the medicine container into the passage formed in the first electrode. A pulse current is caused to flow between the first electrode and second electrode. The liquid medicine flowing through the passage infiltrates into the human skin by iontoforesis. The medicine-infiltratable soft material, being mounted to the first electrode in a freely-detachable manner, can contact the skin smoothly.

### CROSS REFERENCE TO RELATED APPLICATIONS

The Applicant claims priority from his Japanese patent application no. 2001-061888 filed Mar. 6, 2001.

### BACKGROUND OF THE INVENTION

The subject invention relates to iontoforesis devices. Iontoforesis is a method that ionizes medicine, thereby facilitating the penetration of medicine into human skin, etc.

Iontoforesis is a method of administering medicine without pain, wherein medicine is ionized by means of a very weak direct electric current and the ionized medicine is absorbed into human skin.

The usual devices for administering medicine by iontoforesis consist of an electric current generator, conductive gel and the like (acting as an ionic medicine conductive means), a first conductor made of a covered conductor, and a second conductor made of a similar covered conductor and the like. It is not easy to use these usual devices.

In general, the higher the voltage and/or the larger the current becomes, the more satisfactory becomes the absorption of medicine into the skin by means of iontoforesis. However, the user must be careful not to bum or injure his/her skin.

With this in mind, iontoforesis methods using a high-frequency pulse current have been proposed (Japanese Patent Provisional Publications No. 31169/1986 and No. 102768/1988).

Several means have been devised to provide materials capable of containing ionic medicine (gel-layer plaster, etc.) to be used in iontoforesis methods. They include (a) electrodes covered with medicine-infiltrated materials, (b) layers of conductive electrodes and medicine-containing conductive gel, or (c) methods wherein an aqueous solution and medicine are first prepared separately and then are brought into contact with each other at the time of administration (Japanese Patent Provisional Publications No. 317432/1993 and No. 52224/1996).

The above-mentioned usual iontoforesis devices utilize pulsed current, thereby becoming large in size. Thus, they can not be easily used as portable or home-installed devices.

The absorbing power of usual ionic-medicine-containing materials decreases after a lapse of time. Also, medicine-containing conductive gels, etc., are not easy to handle. Further, amounts of absorbed medicines differ according to users and/or applicable skin sections, which makes it difficult to measure the amount of absorbed medicine exactly.

Moreover, there are several different ways of handling medicines in iontoforesis methods. For example, a medicine is applied to the electrode in one method and a medicine is dissolved in water in another method. Also, the use of such medicine requires highly professional knowledge. Thus, general consumers cannot use such medicines. Also, no easy-to-use devices or medicines are available to general consumers.

Development of a portable iontoforesis device that can be used by everybody anytime anywhere represents a great improvement in the field of medicine and satisfies a long felt need of the general consumers.

### SUMMARY OF THE INVENTION

In order to solve the problems identified above, the subject invention proposes iontoforesis devices whose output between the electrodes is of low impedance and whose structures have (a) an iontoforesis power-source unit consisting of a power source, pulse generating circuit and the like, (b) a first electrode having a passage for supplying medicine, etc., (c) a section capable of holding a detachable medicine container that can be freely connected to the passage, and (d) an second electrode that is connected to the iontoforesis power source unit and is exposedly mounted on one end of the iontoforesis device. Thus, the device can be made small and light; it can be designed so that everyone can use it easily anywhere; no general knowledge of medicine is required of users; and it can be designed so that users can readily use it for their respective purposes after they are given a brief explanation of use.

It is the object of this invention to provide an iontoforesis device having (a) an iontoforesis power source unit consisting of a power source, pulse generating circuit and the like, (b) a first electrode having a passage for supplying medicine, etc., (c) a section capable of holding a detachable medicine container that can be freely connected to the passage, and (d) a second electrode that is connected to the iontoforesis power source unit and is exposedly mounted on one end of the iontoforesis device. Thus, the device can be made small, light and portable. Also, as the medicine container can be easily attached/detached anytime anywhere, there is no need to use antiseptic since a fresh medicine container can be attached at the time of use. Also, a medicine container of a specific shape can be used for a specific medicine, thereby avoiding the danger of using the wrong medicine.

The above-mentioned structure and features can allow only the medicines that are controlled by the medicine provider to be used. Thus, iontoforesis can be used effectively and safely anytime and anywhere, thereby treatment at home will be possible.

It is another object of this invention to provide a freely openable-closable cover, mounted on the main body so that the medicine container is pressed by the cover, thereby liquid medicine will be injected through the injection port. This design allows medicine to be smoothly supplied from the container without being touched by the users' hand.

It is a further object of this invention to make the first electrode of a solid conductive material with a surface covered with a medicine-infiltratable soft material, so that there is no need to replace electrodes and the medicine-infiltratable soft material is soft to the skin and easy to use.

It is a still further object of this invention to provide a window in the cover through which the medicine-infiltratable soft material is exposed while it is pressed and held by the cover. Consequently, the medicine-infiltratable soft material can be easily replaced every time of use so that a clean material can be used at all times.

It is a still further object of this invention to provide a medicine-holding section formed in the main body corresponding to the shape of the medicine container. Thus, the medicine supplied can be specified or distinguished by the shape of the medicine containers. Thus the iontoforesis power source can be matched with the medicine supplied and the use of medicine can be controlled even if the administrator is not present at the site of use.

An appreciation of the other aims and objectives of the present invention and an understanding of it may be achieved by referring to the accompanying drawings and description of a preferred embodiment.

### BRIEF DESCRIPTION OF THE DRAWINGS

FIG. 1 is a side view of the working end of the invention with the cover open.

FIG. 2 is cross sectional view of the first electrode and medicine container.

FIG. 3 a side view of the working end of the invention with the cover closed.

FIG. 4 is a partially cut away bottom view of the working end of the invention.

FIG. 5 is a schematic diagram of the power source of the invention

FIG. 6 is a side view the preferred, hand-held embodiment of the invention.

### DETAILED DESCRIPTION OF THE PREFERRED EMBODIMENT

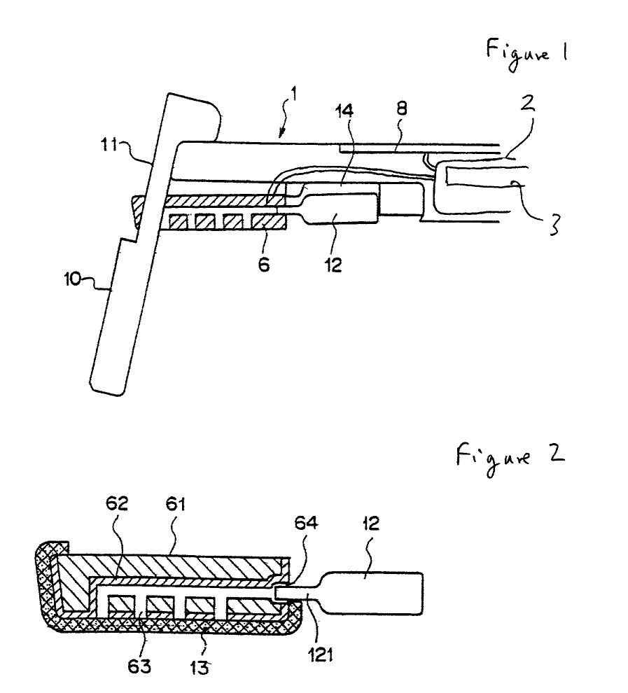

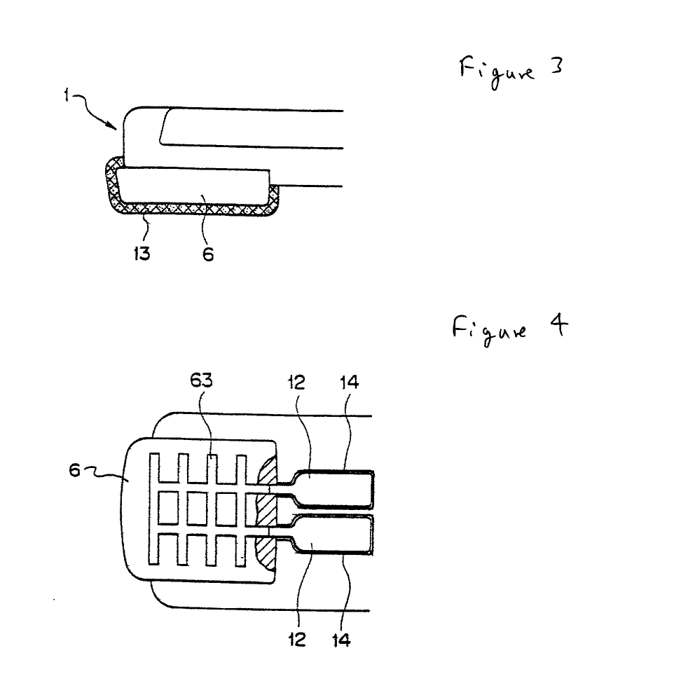

Referring to FIGS. 1-6, the subject invention is explained in detail below.

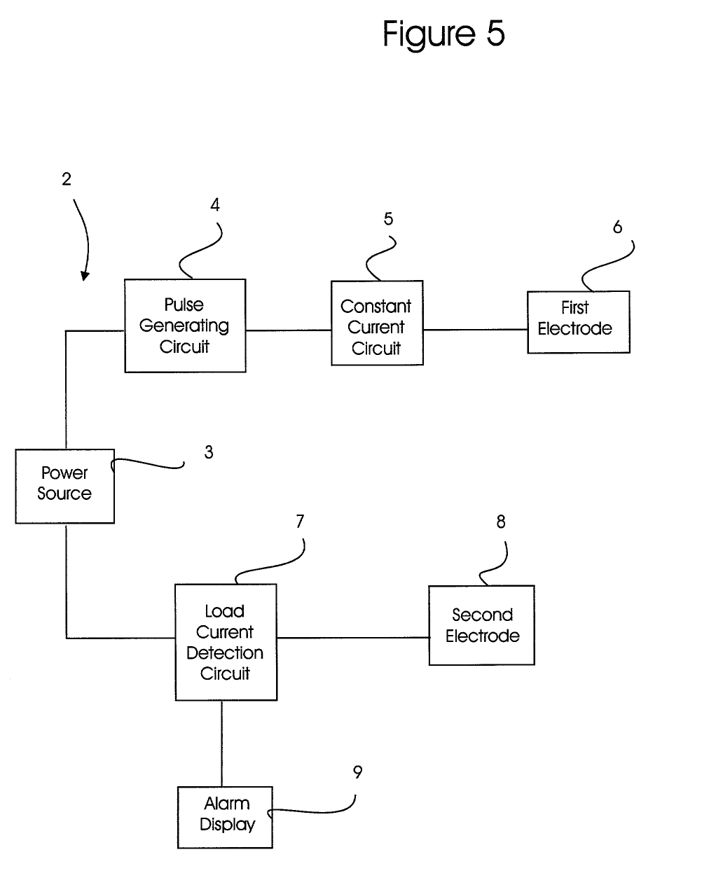

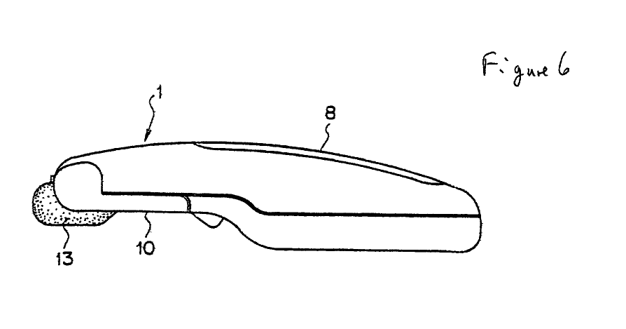

1 indicates the main body of the device. It is preferably made of plastic. The iontoforesis power source unit 2 is incorporated inside the main body 2. The iontoforesis power source unit 2 is structured as shown in FIG. 5. Namely a power source 3 is connected to a pulse generating circuit 4 that is connected to a constant power source circuit 5 that is then connected to first electrode 6. Preferably, the power source 3 is a battery. Also, the power source 3 is connected to the load current detection circuit 7 that is then connected to the second electrode 8. Further, an alarm display 9 can be connected to the load current detection circuit 7 when necessary. The load current detection circuit 7 detects variations in amperage and voltage and sends a signal to the alarm display 9.

Iontoforesis is a method of causing medicine to be infiltrated and absorbed into the human body by applying a first electrode 6 containing medicine and a second electrode 8 containing no medicine to the human skin, etc., and by causing an electric current to flow between the electrodes 6, 8. Direct current or pulse is used to energize the electrodes 6, 8. If pulse is used, a pulse duty ratio is set to shorten the pulse-on time and lengthen the pulse-off time. Also, the current stored in the polarization capacity can be discharged by short-circuiting the first electrode 6 and second electrode 8 during the period when pulse is at rest (the depolarization discharging method). Use of such methods can provide smaller and lighter iontoforesis power source units. For that, it is recommended that the frequency be 1 kHz or less.

If the main body 1 is made in a flat shape (see FIG. 6), it can be easily operated by hand. A cover 10 is mounted on one end of main body 1 in a freely openable-closable manner. The first electrode 6 is mounted on main body 1 corresponding to an opening 11 in the cover 10. The first electrode 6 is preferably made of plastic 61 with its surface plated with an evaporated metal 62 (such as copper or nickel). Liquid medicine passages 63 are formed in the first electrode 6. The first electrode 6 also has and injection port 64 connected to the liquid medicine passages 63 to which a liquid medicine container 12 can be freely attached. The liquid medicine passages 63 can be formed vertically, horizontally or angled so that liquid medicine can flow well. Preferred shapes include channels and pipes.

Injection port 64 is made in such a shape that mouth 121 of liquid medicine container 12 can be freely inserted into it. Liquid medicine container 12 may be inserted through the injection port 64 with its mouth 121 cut off. Alternatively, a needle-like protrusion may be formed at the injection port 64 so that it will make a hole at the tip of the mouth 121 when it is inserted into the injection port 64. Recesses 14 are formed in the main body 1 to hold liquid medicine container 12. Also, the invention is preferably designed so that the cover 10 presses on the liquid medicine container 12 when the cover 10 is closed.

A medicine infinitrable material 13 covers the first electrode 6. Any material that allows liquid medicine to infiltrate into it and is soft to the skin can be used such as cotton nonwoven fabric, porous film and the like. The cover 10 has a window 11. When the cover 10 is closed, the center of the material 13 is exposed through the window while the edges of the material are pressed and held by cover 10.

A second electrode 8 is mounted on the other end of main body 1 opposite the cover 10 in such a manner that, when the user holds the main body 1, his/her hand touches the second electrode 8. The second electrode 8 can be made of stainless steel or plastic plated with an evaporated metal. Also, polarized electrodes, such as platinum, titanium, carbon, etc., can be used for first electrode 6 and second electrode 8. Preferably a depolarization circuit is inserted between the first electrode 6 and the second electrode 8. A depolarization unit reduces the intensity of the electrical charge. The unit operates best within a range of 0.2 to 1.0 5 milliamps at 5 to 6 volts. The pulse cycle is 0.6 milliseconds on and 0.4 milliseconds off. If the current goes outside of the preferred range of 0.2 to 1.0 milliamps, the alarm 9 advises the user of this, preferably by making a beep for 15 seconds.

The liquid medicine container 12 can be a standard medical ampoule, container, bag, etc., made of soft plastic, etc. Preferably, the liquid medicine container 12 has the specific and exclusive shape that matches the shape of the injection port 121 and the section 14 of the main body 1 that holds the liquid medicine container 12.

Medicines of different types can be mixed. Also, more than one liquid medicine container 12 can be used. See FIG. 4. A wide variety of medicines can be used for the subject invention; however, home-use drugs such as vitamins, minerals (magnesium, zinc, selenium, etc.), placenta, etc., in particular can be effectively used.

Use of the device by this invention is explained below.

The cover 10 is opened and a liquid medicine container 12 is set to the holding section 14. At the same time, the mouth 121 of the liquid medicine container 12 is inserted into the injection port 64 with the mouth 121 opened (by cutting off, making a hole to or unsealing the tip of mouth 121). Liquid medicine then flows from the liquid medicine container 12 through its mouth 121 and via the injection port 64 into the liquid medicine passages 63 of the first electrode 6. The medicine then infiltrates into the material 13.

Then, power is turned on to the iontoforesis power source unit 2 so the pulse generating circuit 4, constant power source circuit 5 and then first electrode 6 are energized. Also, the power source 3 energizes the second electrode 8 via load current detection circuit 7. When the user holds the main body 1, his/her hand touches second electrode 8 and when the material 13 is applied to a desired section of the skin, an electric current flows between both electrodes. Thus, a conductive circuit is formed between first electrode 6 and the skin. At this time, the liquid medicine infiltrated into the material 13 becomes ionized and capable of electrical administration through the skin. Thus, the ionized medicine is absorbed into the body through the skin.

The following reference numerals are used on FIGS. 1 through 6:

1. Main body

2. Power source unit

3. Battery

4. Pulse generating circuit

5. Constant power source circuit

6. First electrode

7. Load current detection circuit

8. Second electrode

9. Alarm display

10. Cover

11. Window

12. Liquid medicine container

13. Medicine infinitrable material

14. Recess for holding liquid medicine container

61. Plastic base of first electrode

62. Metal covering of first electrode

63. Liquid medicine passage

64. Injection port

121. Mouth of liquid medicine container

This iontoforesis device has been described with reference to a particular embodiment. Other modifications and enhancements can be made without departing from the spirit and scope of the claims that follow.

What is claimed is:

1. An iontoforesis device comprising:
a. a main body having a section capable of holding a detachable liquid medicine container;
b. a direct power source contained in said main body;
c. a first electrode, mounted at one end of said main body, electrically connected to said pulsed, direct power source;
d. a medicine-infiltratable soft material covering said first electrode; said first electrode and adapted to convey liquid medicine from said detachable liquid medicine container to said medicine-infiltrable soft material; and
e. a second electrode, electrically connected to said direct power source, exposedly mounted at the other end of said main body.

2. An iontoforesis device as claimed in claim 1 in which said power source provides 0.2 to 1.0 milliamps at 5 to 6 volts.

3. An iontoforesis device as claimed in claim 1 in which said power source is pulsed.

4. An iontoforesis device as claimed in claim 3 in which the pulse cycle is is 0.6 milliseconds on and 0.4 milliseconds of.

5. An iontoforesis device as claimed in claim 1 further comprising an alarm connected to said direct power source; said alarm set to notify if the current goes outside of the range of 0.2 to 1.0 milliamps.

6. An iontoforesis device claimed in claim 1 further comprising a freely openable-closable cover adapted to apply pressure to said liquid medicine container.

7. An iontoforesis device claimed in claim 6 in which said cover has window adapted to expose the center of said medicine-infiltratable soft material while said cover holds the edges of said medicine-infiltratable soft material.

8. An iontoforesis device comprising:
a. a main body having a section capable of holding a detachable liquid medicine container;
b. an iontoforesis power source unit contained in said main body comprising:
a direct current power source, a pulse generating circuit, a constant current circuit, and a load detection circuit, all interconnected;
c. a first electrode having an interconnected injection port and a passage for supplying liquid medicine, mounted at one end of said main body, adjacent said section capable of holding said detachable liquid medicine container; said injection port capable of connecting to said detachable liquid medicine container; said first electrode being electrically connected to said iontoforesis power source unit;
d. a medicine-infiltratable soft material covering said first electrode; and
e. a second electrode, electrically connected to said iontoforesis power source unit, exposedly mounted at the other end of said main body.

9. An iontoforesis device as claimed in claim 8 in which said power source provides 0.2 to 1.0 milliamps at 5 to 6 volts.

10. An iontoforesis device as claimed in claim 8 in which said power source is pulsed.

11. An iontoforesis device as claimed in claim 10 in which the pulse cycle is 0.6 milliseconds on and 0.4 milliseconds of.

12. An iontoforesis device as claimed in claim 8 further comprising an alarm connected to said direct power source; said alarm set to notify if the current goes outside of the range of 0.2 to 1.0 milliamps.

13. An iontoforesis device claimed in claim 8 further comprising a freely openable-closable cover adapted to apply pressure to said liquid medicine container.

14. An iontoforesis device claimed in claim 13 in which said cover has window adapted to expose the center of said medicine-infiltratable soft material while said cover holds the edges of said medicine-infiltratable soft material.

15. An method of administering medicine comprising the steps of:
a. Providing an iontoforesis device comprising:
i. a main body having a section capable of holding a detachable liquid medicine container;
ii. an iontoforesis power source unit contained in said main body comprising: a direct current power source, a pulse generating circuit, a constant current circuit, and a load detection circuit, all interconnected;
iii. a first electrode having an interconnected injection port and a passage for supplying liquid medicine, mounted at one end of said main body, adjacent said section capable of holding said detachable liquid medicine container; said injection port capable of connecting to said detachable liquid medicine container; said first electrode being electrically connected to said iontoforesis power source unit;
iv. a medicine-infiltratable soft material covering said first electrode;
v. a second electrode, electrically connected to said iontoforesis power source unit, exposedly mounted at the other end of said main body; and
vi. a freely openable-closable cover, attached to said main body, adapted to apply pressure to said liquid medicine container; said cover having a window adapted to expose the center of said medicine-infiltratable soft material while said cover holds the edges of said medicine-infiltratable soft material;
b. placing said detachable liquid medicine container in said section while inserting the mouth of said liquid medicine container in said injection port;
c. closing said cover, whereby liquid medicine infiltrates said medicine-infiltrable soft material and said;
d. holding said main body so that one hand contacts said second electrode;
e. placing said first electrode covered with said medicine infiltrated soft material on the skin; and
f. activating said ionotoforesis power source unit, whereby said liquid medicine becomes ionized and is absorbed through the skin.

16. A method as claimed in claim 15 further comprising the step of controlling said power source to provide 0.2 to 1.0 milliamps at 5 to 6 volts.

17. A method as claimed in claim 15 further comprising the step of pulsing said power source.

18. A method as claimed in claim 15 further comprising the step of controlling the pulse cycle to 0.6 milliseconds on and 0.4 milliseconds off.

19. A method as claimed in claim 15 further comprising the steps of:
a. connecting an alarm to said direct power source; and
b. setting said alarm to notify if the current goes outside of the range of 0.2 to 1.0 milliamps.
